(19) United States
(12) Patent Application Publication
(10) Pub. No.: US 2009/0093625 A1
(43) Pub. Date: Apr. 9, 2009

(54) APPARATUS FOR AND METHOD OF SYNTHESIZING BIOPOLYMER AND METHOD OF RECOVERING REAGENT FOR SYNTHESIZING BIOPOLYMER

(75) Inventors: Sung-Min Chi, Gyeonggi-do (KR); Man-Hyoung Ryoo, Gyeonggi-do (KR); Hyeong-Jun Kim, Seoul (KR); Jung-Hwan Hah, Gyeonggi-do (KR); Won-Sun Kim, Gyeonggi-do (KR); Sun-Ok Jung, Gyeonggi-do (KR)

Correspondence Address: MARGER JOHNSON & MCCOLLOM, P.C., 210 SW MORRISON STREET, SUITE 400, PORTLAND, OR, 97204, US

(73) Assignee: SAMSUNG ELECTRONICS CO., LTD., Gyeonggi-do (KR)

(21) Appl. No.: 12/238,162

(22) Filed: Sep. 25, 2008

(30) Foreign Application Priority Data
Oct. 4, 2007 (KR) 10-2007-0099991

(51) Int. Cl. C07H 21/04; B01J 19/24

(52) U.S. Cl. 536/25.4; 422/131; 422/135

(57) ABSTRACT

An apparatus for synthesizing a biopolymer includes a reaction chamber, an outlet tube connected to the reaction chamber, a plurality of recovery tanks connected to the outlet tube, and a plurality of recovery valves configured to open or block the passageway between the outlet tube and each of the recovery tanks.

### CROSS-REFERENCE TO RELATED APPLICATION

This application claims the benefit of foreign priority to Korean Patent Application No. 10-2007-0099991, filed on Oct. 4, 2007, the disclosure of which is incorporated herein by reference in its entirety.

### BACKGROUND

1. Field of Invention

Embodiments of the present invention generally relate to apparatuses for synthesizing biopolymers, methods of synthesizing biopolymers and methods of recovering reagents used in synthesizing biopolymers. More particularly embodiments of the present invention relate to apparatus for, and method of synthesizing, a biopolymer in which a reagent used to synthesize the biopolymer can be recovered and recycled. Embodiments of the present invention also relate to a method of recovering a reagent used to synthesize the biopolymer.

2. Description of the Related Art

There is an increasing need for synthesizing polymers on a substrate in various fields including a semiconductor manufacturing field. In particular, microarrays having biopolymers such as oligomer probes fixed onto a slide substrate have been introduced in recent years. Polymer synthesis technology is also being employed to form microarrays.

For example, a photolithographic technique widely used by semiconductor manufacturers may be applied to synthesize oligomer probes in a microarray. The synthesis of oligomer probes using photolithography involves attaching a coupling agent containing a photolabile protecting group onto a substrate, removing the photolabile protecting agent from the coupling agent after selective exposure through a photomask, and providing a monomer to be synthesized so that it can react with the resulting coupling agent.

To synthesize 25-mer oligomer probes, the synthesis step is repeated 25 to 100 times. A reaction yield at each step between a monomer to be synthesized and a coupling agent may significantly affect the overall processing yield. Thus, there is an urgent need to maximize the reaction yield at each synthesis step.

### SUMMARY

Embodiments of the present invention can be generally characterized as capable of providing an apparatus for synthesizing a biopolymer, by which a reagent for synthesizing the biopolymer can be recovered and recycled.

Embodiments of the present invention can be generally characterized as capable of providing a method of recovering a reagent for synthesizing the biopolymer, by which the cost required for synthesizing the biopolymer can be reduced.

Embodiments of the present invention can be generally characterized as capable of providing a method of synthesizing a biopolymer, by which a reagent for synthesizing the biopolymer can be recovered and recycled.

The above and other embodiments of the present invention will be described in or be apparent from the following description of the preferred embodiments.

One embodiment of the present invention can be generally characterized as an apparatus for synthesizing a biopolymer. The apparatus may include a reaction chamber, an outlet tube connected to the reaction chamber, a plurality of recovery tanks connected to the outlet tube, and a plurality of recovery valves configured to open or block passageways between the outlet tube and corresponding ones of the recovery tanks.

Another embodiment of the present invention can be generally characterized as a method of recovering a biopolymer synthesis reagent. The method may include supplying a first biopolymer synthesis reagent to a reaction chamber in which a substrate is seated and synthesizing the first biopolymer synthesis reagent on the substrate, wherein an amount of first biopolymer synthesis reagent may remain within the reaction chamber; recovering the amount of first biopolymer synthesis reagent in a first recovery tank via an outlet tube; cleaning the reaction chamber and the outlet tube using cleaning solution; supplying a second biopolymer synthesis reagent to the reaction chamber and synthesizing the second biopolymer synthesis reagent on the substrate, wherein an amount of second biopolymer synthesis reagent may remain within the reaction chamber; and recovering the amount of second biopolymer synthesis reagent in a second recovery tank via the outlet tube.

Yet another embodiment of the present invention can be generally characterized as a method of recovering a biopolymer synthesis reagent. The method may include performing a first biopolymer synthesis and recovery cycle; performing a second biopolymer synthesis and recovery cycle; and performing a cleaning cycle after the first biopolymer synthesis and recovery cycle and before the second biopolymer synthesis and recovery. The first biopolymer synthesis and recovery cycle may be performed by a method that includes supplying a first biopolymer synthesis reagent to a reaction chamber in which a substrate is seated and synthesizing the first biopolymer synthesis reagent on the substrate, wherein an amount of first biopolymer synthesis reagent remains within the reaction chamber; and recovering the amount of first biopolymer synthesis reagent in a first recovery tank selected from a plurality of recovery tanks via an outlet tube. The second biopolymer synthesis and recovery cycle may be performed by a method that includes supplying a second biopolymer synthesis reagent to a reaction chamber in which a substrate is seated and synthesizing the second biopolymer synthesis reagent on the substrate, wherein an amount of second biopolymer synthesis reagent remains within the reaction chamber; and recovering the amount of second biopolymer synthesis reagent in a second recovery tank selected via the outlet tube. The cleaning cycle may include cleaning the reaction chamber and the outlet tube using a cleaning solution. Each of the first and second biopolymer synthesis and recovery cycles may be performed two or more times. The first biopolymer synthesis reagent produced after the first cycle of the two or more first synthesis and recovery cycles may include the first biopolymer synthesis reagent returned from the first recovery tank and the second biopolymer synthesis reagent produced after the first cycle of the two or more second synthesis and recovery cycles may include the second biopolymer synthesis reagent returned from the second recovery tank.

### BRIEF DESCRIPTION OF THE DRAWINGS

The above and other embodiments of the present invention will become more apparent by describing in detail preferred embodiments thereof with reference to the attached drawings in which:

FIG. 1 is a schematic diagram of an apparatus for synthesizing a biopolymer according to an embodiment of the present invention;

FIG. 2 is a cross-sectional view of a reaction chamber according to an embodiment of the present invention;

FIG. 3 is a plan view illustrating a shaking apparatus and a reaction chamber according to an embodiment of the present invention;

FIG. 4 is a front view of the shaking apparatus and reaction chamber shown in FIG. 3;

FIG. 5 is a side view illustrating a shaking operation of the apparatus for synthesizing a biopolymer according to an embodiment of the present invention;

FIG. 6 is a cross-sectional view of a modified exemplary reaction chamber according to an embodiment of the present invention;

FIG. 7 is a flowchart illustrating a method of synthesizing a biopolymer according to an embodiment of the present invention;

FIGS. 8 through 14 are sectional views of processing steps illustrating a method of synthesizing the biopolymer according to an embodiment of the present invention;

FIG. 15 is a schematic diagram of an apparatus for synthesizing a biopolymer according to another embodiment of the present invention; and

FIG. 16 is a flowchart illustrating a method of synthesizing a biopolymer according to another embodiment of the present invention.

### DETAILED DESCRIPTION

Embodiments of the present invention may be understood more readily by referring to the following detailed description and the accompanying drawings. These embodiments may, however, be realized in many different forms and should not be construed as being limited to the disclosure set forth herein. Rather, these embodiments are provided so that this disclosure will be thorough and complete and will fully convey the concept of the invention to those skilled in the art, and the present invention will only be defined by the appended claims. Like reference numerals refer to like elements throughout the specification.

In some embodiments, well-known process procedures, structures, and techniques will not be described in detail to avoid misinterpretation of the present invention.

The terminology used herein is for the purpose of describing particular embodiments only, and is not intended to be limiting of the invention as recited in the claims. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Target biopolymers that will be synthesized on a substrate contain polymers that are synthesized within or construct a living body. Biopolymers are composed of two or more monomers. Some examples of monomers may be nucleosides, nucleotides, amino acids, peptides, etc., according to the type of probes.

As used herein, the terms “nucleosides” and “nucleotides” include not only known purine and pyrimidine bases, but also methylated purines or pyrimidines, acylated purines or pyrimidines, etc. Furthermore, the “nucleosides” and “nucleotides” include not only known (deoxy)ribose, but also modified sugars which contain a substitution of a halogen atom or an aliphatic group for at least one hydroxyl group or is functionalized with ether, amine, or the like.

As used herein, the term “amino acids” are intended to refer to not only naturally occurring, L-, D-, and nonchiral amino acids, but also modified amino acids, amino acid analogs, etc.

As used herein, the term “peptides” refers to compounds produced by an amide bond between the carboxyl group of one amino acid and the amino group of another amino acid.

Apparatuses for synthesizing biopolymers according to exemplary embodiments of the present invention will now be described with reference to the accompanying drawings.

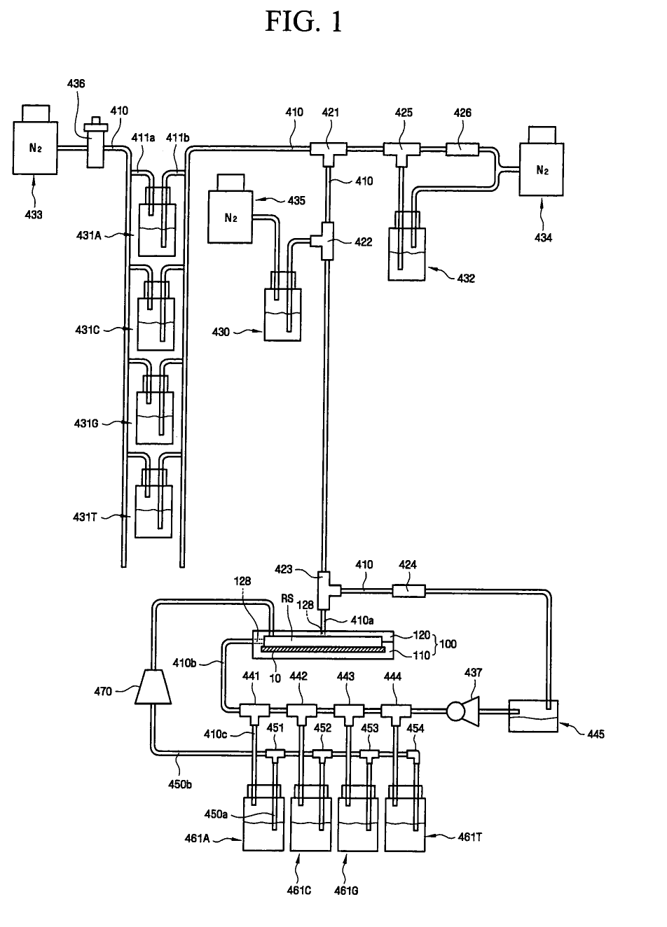

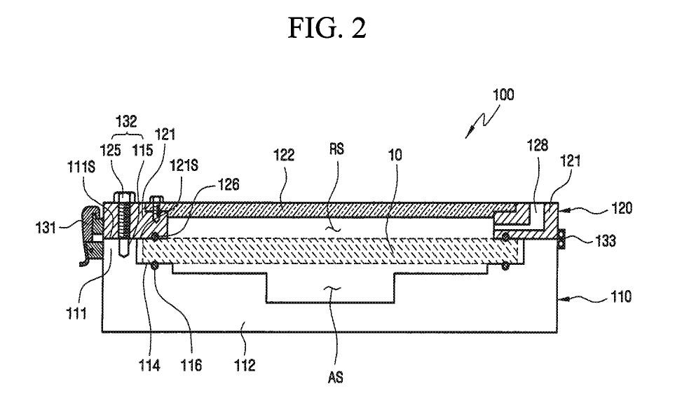

FIG. 1 is a schematic diagram of an apparatus for synthesizing a biopolymer according to an embodiment of the present invention. FIG. 2 is a cross-sectional view of a reaction chamber according to an embodiment of the present invention.

Referring to FIGS. 1 and 2, a biopolymer synthesis apparatus according to an embodiment of the present invention may, for example, include a reaction chamber 100, a plurality of recovery tanks (e.g., first recovery tank 461A, second recovery tank 461C, third recovery tank 461G and fourth recovery tank 461T), and an outlet tube 410b. A substrate 10, on which biopolymers are to be synthesized, may be disposed within the reaction chamber 100.

Within the reaction chamber 100, target biopolymers are synthesized by sequentially forming a covalent bond between monomeric units on a substrate 10. In another embodiment, however, target biopolymers may be synthesized by creating a covalent bond between a biopolymer formed from at least two monomers covalently bonded together and another monomer or biopolymer on the substrate 10.

The substrate 10 can be either flexible or rigid. A flexible substrate may be a membrane such as nylon or nitrocellulose or plastic film. A rigid substrate may be a semiconductor wafer substrate or transparent glass substrate such as soda-lime glass, or the like. For effective biopolymer synthesis, monomer, biopolymer, or other organic or inorganic linkers may be fixed on the substrate 10.

The shape and size of the reaction chamber 100 may vary according to the shape and size of the substrate 10 disposed therein. For example, if a circular silicon wafer is used as the substrate 10, the reaction chamber 100 may have a cylindrical shape.

In the illustrated embodiment, the reaction chamber 100 includes a chamber body 110 and a chamber cover 120. The chamber cover 120 is coupled to the chamber body 110. A detailed explanation regarding shape of the reaction chamber 100 and its engagement with other components is provided with respect to FIG. 2.

Referring to FIG. 2, the chamber body 110 is coupled to the chamber cover 120 by, for example, a first coupling unit 131. In one embodiment, the first coupling unit 131 is a clamp. A plurality of clamps may be arranged along an outer perimeter of the chamber body 110 and/or chamber cover 120. In some exemplary embodiments, a connecting pin 133 is located at one rim of the chamber body 110 and the chamber cover 120.

As illustrated in FIG. 2, the chamber body 110 has a step height difference between a rim 111 and a central portion 112 thereof. The chamber cover 120 also has a step height difference between a rim 121 and a central portion 122 thereof. Accordingly, the chamber body 110 and the chamber cover 120 have rims 111 and 121, respectively, protruding beyond respective central portions 112 and 122, to form a container. The rims 111 and 121 of the chamber body 110 and the chamber cover 120, respectively, may have substantially planar surfaces 111S and 121S. Thus, when the chamber body 110 is coupled to the chamber cover 120, the surface 111S of the rim 111 of the chamber body 110 contacts the surface 121S of the rim 121 of the chamber cover 120, while the central portion 112 of the chamber body 110 is spaced apart from the central portion of the chamber cover 120.

According to one exemplary embodiment, the reaction chamber 100 may further include second coupling units 132 disposed at the rims 111 and 121 of the chamber body 110 and the chamber cover 120. The second coupling unit 132 may, for example, include a screw 125 protruding from the rim 121 of the chamber cover 120 and extending into a fitting hole 115 recessed into the rim 111 of the chamber body 110.

A substrate seating end 114 is disposed inside the rim 111 of the chamber body 110. A predetermined air space (AS) is defined between the substrate 10 placed on the substrate seating end 114 and the central portion 112 of the chamber body 110.

When seated on the substrate seating end 114, the substrate 10 is spaced apart from the central portion 122 of the chamber cover 120 by the height of the rim 121 of the chamber cover 120 projecting out from the central portion 122, thereby defining a reaction space (RS) in which biopolymers can be synthesized. Accordingly, the reaction space RS is defined by the central portion 122 of the chamber cover 120, a side wall of the rim 121 of the chamber cover 120 and a top surface of the substrate 10. In order to facilitate observation of various reactions that may occur within the reaction space RS, at least the central portion 122 of the chamber cover 120 may be formed of a transparent material such as glass or quartz. Accordingly, a window may be provided at the central portion 122 of the chamber cover 120.

The spreadability and wettability of the reagent affects the amount of reagent that can be used for synthesis of biopolymers. In addition, the size (volume) of the reaction space RS affects the amount of reagent that can be used for synthesis of biopolymers, and varies depending on a distance between the central portion 122 of the chamber cover 120 and the top surface of the substrate 10 (i.e., the height of the rim 121 protruding from the central portion 122 of the chamber cover 120).

As described above, because the reaction space RS is created within a sealed interior space in the reaction chamber 100, the reaction space RS is also substantially sealed from the outside. Further, since the top edge of the substrate 10 is closely sealed by the rim 121 of the chamber cover 120 and the rear surface of the substrate 10 is supported by the substrate seating end 114, the air space AS defined between the rear surface of the substrate 10 and the central portion 112 of the chamber body 110 is spatially separated from the reaction space RS. Accordingly, the reaction space RS is substantially sealed from the air space AS. Thus, when a biopolymer synthesis reagent is introduced into the reaction space RS, the biopolymer synthesis reagent does not infiltrate onto the rear surface of the substrate 10 and contamination on the rear surface of the substrate 10 is prevented. It is desirable to prevent contamination of the rear surface of the substrate 10 because such contamination may result in an error in analysis of biomaterials or result in a malfunction in a photolithography apparatus that is subsequently used.

To further ensure prevention of contamination of the rear surface of the substrate 10, the reaction chamber 100 may, in one exemplary embodiment, further include a gasket disposed on the substrate seating end 114 of the chamber body 110 and/or the rim 121 of the chamber cover 120. For example, an O-ring 116 or 126 may be used as the gasket. The O-ring 116 disposed on the substrate seating end 114 of the chamber body 110 and the O-ring 126 disposed on the rim 121 of the chamber cover 120 may directly contact the rear and top surfaces of the substrate 10, respectively, to reliably prevent infiltration of a fluid such as a reagent for polymer synthesis into the air space AS.

Referring to FIG. 1, the reaction space RS is spatially connected to one or more of the supply tube 410a, outlet tube 410b and recovery tubes 410c. To achieve this, at least one of the chamber body 110 and the chamber cover 120 includes a plurality of through holes 128, wherein each of the plurality of through holes 128 is coupled to a corresponding one of the supply tube 410a, the outlet tube 410b and the recovery tubes 410c. In one embodiment, each through hole 128 may have one end that opens at a sidewall of the rim 121 of the chamber cover 120 and another end that is coupled to the supply tube 410a, the outlet tube 410b or a recovery tube 410c. A biopolymer synthesis reagent, an activator and inactive gas are introduced into (or discharged out of) the reaction space RS through the supply tube 410a (or outlet tube 410b) and a corresponding through hole 128. Some of the one or more supply tubes 410a may be dedicated to conveying inactive gas. In addition, biopolymer synthesis reagent which has been activated by the activator (i.e., activated biopolymer synthesis reagent) is returned from recovery tanks 461A, 461C, 461G and 461T to the reaction space RS through a return tube 450b.

In addition to the supply tube 410a, the outlet tube 410b and the recovery tubes 410c, the biopolymer synthesis apparatus according to an embodiment of the present invention may further include a cleaning tank 430, a plurality of reagent tanks (e.g., first reagent tank 431A, second reagent tank 431C, third reagent tank 431G and fourth reagent tank 431T), an activator tank 432, a plurality of fluid flow tubes 410 and a plurality of valves (e.g., first valve 421, second valve 422, third valve 423, fourth valve 424, fifth valve 425 and sixth valve 426) connecting the fluid flow tubes 410. In addition to the recovery tanks 461A, 461C, 461G and 461T, the biopolymer synthesis apparatus may include a plurality of recovery valves (e.g., first recovery valve 441, second recovery valve 442, third recovery valve 443 and fourth recovery valve 444), a plurality of return valves (e.g., first return valve 451, second return valve 452, third return valve 453 and fourth return valve 454) and a return pump 470.

The plurality of reagent tanks 431A, 431C, 431G and 431T store reagents needed for synthesis of biopolymers and provide the reagents to the reaction space RS within a reaction chamber 100 via the supply tube 410a. Examples of biopolymer synthesis reagents that may be provided by the reagent tanks 431A, 431C, 431G and 431T include monomers such as nucleoside, nucleotide, amino acid, or peptide as described above and compounds thereof. For example, if oligonucleotide probes are synthesized in situ, the biopolymer synthesis reagent may be a nucleotide phophoramidite monomer having a base that is one of Adenine (A), Thymine (T), Guanine (G), Cytosine (C) and Uracil (U) and photolabile or acid labile protecting groups coupled thereto. The biopolymer synthesis reagents stored in the respective reagent tanks may have different bases. Accordingly, the first reagent tank 431A may store a first biopolymer synthesis reagent having an Adenine (A) base, the second reagent tank 431C may store a second biopolymer synthesis reagent having a Cytosine (C) base, the third reagent tank 431G may store a third biopolymer synthesis reagent having a Guanine (G) base and the fourth reagent tank 431T may store a fourth biopolymer synthesis reagent having a Thymine (T) or Uracil (U) base. As used herein, the term “biopolymer synthesis reagent” refers to a raw material reagent for biopolymer synthesis and also a recycled reagent for biopolymer synthesis.

The cleaning tank 430 may include a cleaning solution (e.g., an acetonitrile solution) used to remove biopolymer synthesis reagent in the supply tube 410a, the outlet tube 410b, the recovery tubes 410c, sub-return tubes 450a and the return tube 450b. Biopolymer synthesis reagent in the supply tube 410a, the outlet tube 410b, the recovery tubes 410c, the sub-return tubes 450a and the return tube 450b may also be removed by an inactive gas (e.g., nitrogen, N₂) provided from a second inactive gas supply tank 434. Accordingly, the supply tube 410a, the outlet tube 410b, the recovery tubes 410c, the sub-return tubes 450a and the return tube 450b may be dried by the inactive gas provided by a second inactive gas supply tank 434.

The activator tank 432 may provide an activator for activating biopolymer synthesis. The activator may be a tetrazole-based activator, for example, 1H-tetrazole or derivatives thereof.

First, second and third inactive gas supply tanks 433, 434 and 435, respectively, may supply an inactive gas such as nitrogen (N₂). Gas supplied from the first inactive gas supply tank 433 is introduced into the reagent tanks 431A, 431C, 431G and 431T via the fluid flow tube 410 and sub-fluid flow tubes 411a provided at the respective reagent tanks 431A, 431C, 431G and 431T, and is then used to apply a predetermined pressure to the reagent tanks 431A, 431C, 431G and 431T, thus allowing the biopolymer synthesis reagent to be pushed up toward the fluid flow tube 410 via a sub-fluid flow tube 411b. Only one biopolymer synthesis reagent having bases to be synthesized stored in a reagent tank among the reagent tanks 431A, 431C, 431G and 431T is first introduced into the reaction chamber 100 via the sub-fluid flow tube 411b for synthesis of some biopolymers. When necessary, the respective reagent tanks 431A, 431C, 431G and 431T, which store biopolymer synthesis reagents having different bases to be synthesized, may be opened for synthesis of other biopolymers. The fluid flow system may further include a pressure controller 436 that is disposed between the first inactive gas supply tank 433 and the respective reagent tanks 431A, 431C, 431G and 431T and appropriately adjusts the pressure therebetween.

Inactive gas supplied by the second inactive gas supply tank 434 is introduced into the activator tank 432 through fluid flow tube and is then used to apply a predetermined pressure to the activator tank 432, thus allowing the activator to be pushed up toward the fluid flow tube 410. In addition, the third inactive gas supply tank 435 supplies inactive gas to the cleaning tank 430 to transfer cleaning solution within the cleaning tank 430 toward the fluid flow tube 410.

The plurality of valves 421, 422, 423, 424, 425 and 426 may include at least one of a 3-way solenoid valve and a 2-way solenoid valve. For example, the valves 421, 422, 423 and 425 may include a 3-way solenoid valve and the valves 424 and 426 may include a 2-way solenoid valve. Although not shown, the 3-way solenoid valve may be provided between the fluid flow tube 410 and each of the sub-fluid flow tubes 411a and 411b. The cleaning tank 430, the reagent tanks 431A, 431C, 431G and 431T, and the activator tank 432 are connected to the first, second and third inactive gas supply tanks 433, 434 and 435, the reaction chamber 100 and a drain 445.

In addition to the fluid flow tube 410, the outlet tube 410b and return tube 450b are connected to the reaction chamber 100.

The outlet tube 410b has one end connected to the reaction chamber 100 and the other end connected to the drain 445. A drain pump 437 may further be installed at the outlet tube 410b.

The plurality of recovery tubes 410c are branched from the outlet tube 410b and are connected to the plurality of recovery tanks 461A, 461C, 461G and 461T.

The plurality of recovery tanks 461A, 461C, 461G and 461T store the biopolymer synthesis reagent recovered from the reaction chamber 100. In embodiments where it is necessary to supply a biopolymer synthesis reagent containing the same base as that contained in the recovered biopolymer synthesis reagent, the recovered biopolymer synthesis reagent is returned to the reaction chamber 100 through the return tube 450b. Accordingly, the biopolymer synthesis reagents containing different bases are independently stored in the respective recovery tanks 461A, 461C, 461G and 461T to be returned to the return tube 450b through corresponding ones of sub-return tubes 450a, connected to the respective recovery tanks 461A, 461C, 461G and 461T. In one embodiment, a return pump 470 may be provided at each return tube 450b.

An example of a fluid flow operation within the fluid flow system having the above-mentioned configuration will now be described. First, when the pressure controller 436 adjusts the pressure to provide inactive gas from the first inactive gas supply tank 433 to the first reagent supply tank 431A, the inactive gas pressurizes a first biopolymer synthesis reagent (e.g., the biopolymer synthesis reagent having an adenine (A) base), thus pushing the first biopolymer synthesis reagent towards the fluid flow tube 410 and the first valve 421. If the first and second valves 421 and 422 are adjusted to establish a passageway to the reaction chamber 100, then the first biopolymer synthesis reagent is introduced into the reaction space RS via the supply tube 410a. Accordingly, the inactive gas can be selectively fed into only the first reagent supply tank 431A by opening only the sub-fluid flow tubes 411a and 411b connected to the first reagent supply tank 431A while blocking the sub-fluid flow tubes 411a and 411b connected to the other reagent tanks 431C, 431G and 431T.

In embodiments where it is desired to provide biopolymer synthesis reagents containing other bases to the reaction chamber 100, inactive gas is supplied by opening only the sub-fluid flow tubes 411a and 411b connected to the reagent tanks 431C, 431G and 431T containing biopolymer synthesis reagents containing other bases, thereby providing only the reagent required for biopolymer synthesis to the reaction chamber 100.

In embodiments where it is desired to sequentially introduce different biopolymer synthesis reagents into the reaction chamber 100 from the respective reagent tanks 431C, 431G and 431T, the cleaning tank 430 cleans the fluid flow tube 410. For example, after the first biopolymer synthesis reagent is introduced from the first reagent tank 431A into the reaction chamber 100 via the fluid flow tube 410, and before a second biopolymer synthesis reagent is supplied to the reaction chamber 100, a residual amount of first biopolymer synthesis reagent in the fluid flow tube 410 and the reaction chamber 100 can be cleaned using the cleaning solution in the cleaning tank 430. Thereafter, a second biopolymer synthesis reagent may be supplied from, for example, the second reagent tank 431C to the reaction chamber 100 the fluid flow tube 410. During the cleaning cycle, only necessary portions are cleaned by adjusting passageways of the valves 421, 422, 423 and 424. In one embodiment, after the cleaning cycle, the reaction chamber 100 may be dried using the inactive gas supplied by the second inactive gas supply tank 434. In addition, to perform the cleaning cycle, the substrate 10 can be temporarily withdrawn from the reaction chamber 100 and then re-inserted after the cleaning cycle has been performed.

If the first biopolymer synthesis reagent and the activator are supplied to the reaction chamber 100, an excessive amount of the activated first biopolymer synthesis reagent may remain in the reaction chamber 100 while adenine (A) bases are coupled to the substrate 10. The remaining activated first biopolymer synthesis reagent may be drained by the outlet tube 410b and recovered by one of the recovery tanks 461A, 461C, 461G and 461T via a corresponding recovery tube 410c by adjusting the passageways of the plurality of recovery valves 441, 442, 443 and 444. For example, the activated first biopolymer synthesis reagent can be recovered in the first recovery tank 461A by opening the first recovery valve 441, and thus opening the passageway to the first recovery tank 461A, while blocking the second, third and fourth recovery valves 442, 443 and 444, and thus blocking the passageways to the other recovery tanks 461C, 461G and 461T. Similarly, other activated biopolymer synthesis reagents can be recovered in recovery tanks 461C, 461G and 461T by adjusting the passageways of the recovery valves 442, 443 and 444, respectively.

As described above, in order to separately recover the respective activated biopolymer synthesis reagents, the number of recovery tanks 461A, 461C, 461G and 461T may be equal to the number of reagent tanks 431A, 431C, 431G and 431T. Thus, various biopolymer synthesis reagents provided from reagent tanks 431A, 431C, 431G and 431T, which are activated in the reaction chamber 100, can then recovered in corresponding ones of the recovery tanks 461A, 461C, 461G and 461T.

Among the activated biopolymer synthesis reagents, the biopolymer synthesis reagent containing bases to be coupled onto the substrate 10 is returned to the reaction chamber 100 using the return pump 470. The respective activated biopolymer synthesis reagents are led to the sub-return tube 450b via the sub-return tubes 450a connected to the respective recovery tanks 461A, 461C, 461G and 461T and are then supplied to the reaction chamber 100. To transfer the activated biopolymer synthesis reagents from the sub-return tubes 450a to the return tube 450b, the passageways of the return valves 451, 452, 453 and 454 are adjusted. The passageways of the return valves 451, 452, 453 and 454 may be adjusted in substantially the same manner in which the passageways of the recovery valves 441, 442, 443 and 444 are adjusted.

The activated biopolymer synthesis reagent introduced into the reaction chamber 100 can be recycled repeatedly a number of times, depending on the concentration and purity of the recycled reagent. The activated biopolymer synthesis reagent having low concentration and purity is drained to the drain 445 using a drain pump 437.

According to the biopolymer synthesis apparatus of the illustrated embodiment, the biopolymer synthesis reagent can be recovered by adjusting the passageways using the return valves 451, 452, 453 and 454. In addition, using the cleaning tank 430 allows different biopolymer synthesis reagents to be recovered and recycled.

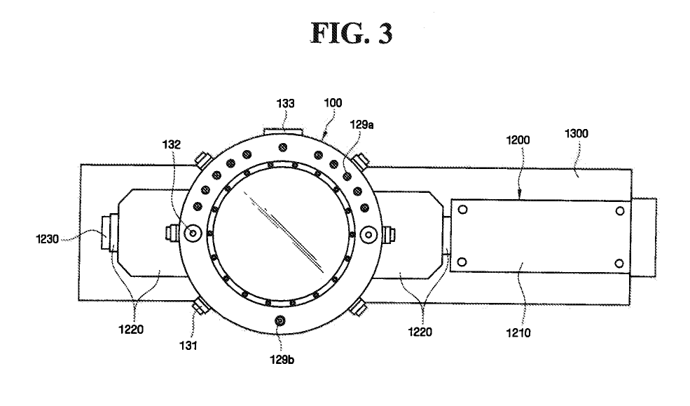

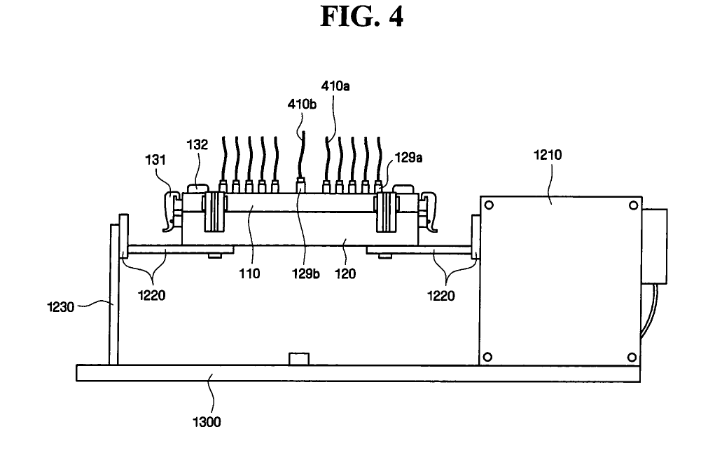

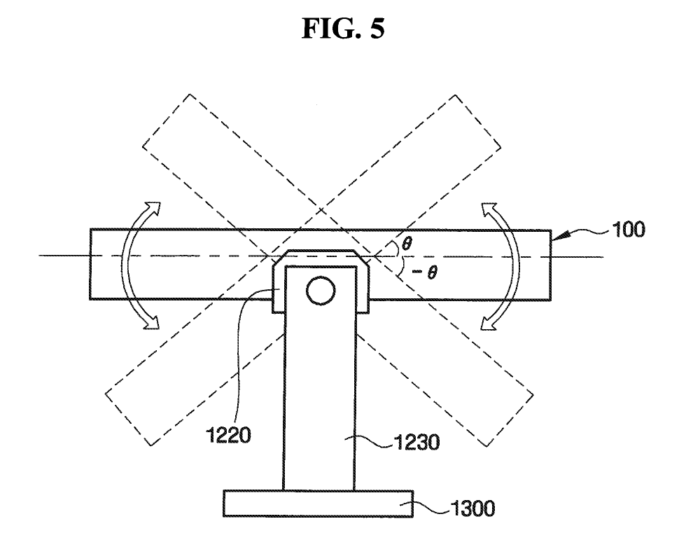

Reference is made to FIGS. 3 through 5 for an exemplary explanation of a shaking unit included in a biopolymer synthesis apparatus of the present embodiment. FIG. 3 is a plan view illustrating a shaking apparatus and a reaction chamber according to an embodiment of the present invention. FIG. 4 is a front view of the shaking apparatus and reaction chamber shown in FIG. 3. FIG. 5 is a side view illustrating a shaking operation of the apparatus for synthesizing a biopolymer according to an embodiment of the present invention.

Referring to FIGS. 3 through 5, the biopolymer synthesis apparatus of the present embodiment may further include a shaking unit 1200. In one embodiment, the shaking unit 1200 includes a drive axis 1220 and a servo motor 1210 driving the drive axis 1220.

The drive axis 1220 has one end connected to the servo motor 1210 and another end connected to a support 1230. The aforementioned reaction chamber 100 may be fixed to the drive axis 1220 (e.g., at the center of the drive axis 1220). The servo motor 1210 and the support 1230 are disposed on a plate 1300.

The servo motor 1210 may rotates the drive axis 1220 and cause the drive axis 1220 to make a rolling motion with a predetermined period. The reaction chamber 100, when fixed to the drive axis 1220, rotates or rolls with the drive axis 1220.

The reaction chamber 100 can rotate while discharging a biopolymer synthesis reagent as will be described below. The maximum angle of rotation by which the reaction chamber 100 rotates may be about ±90°, but the maximum angle of rotation is not strictly limited to the range listed.

The rolling of the reaction chamber 100 can be performed during biopolymer synthesis within the reaction space RS. The maximum angle ±θ that the reaction chamber 100 rolls may vary depending on the amount of biopolymer synthesis reagent contained in the reaction space RS, but can typically be in a range of between about ±10° (i.e., rolling at angles from about −10° to about +10°) and about ±60° (rolling at angles from about −60° to about +60°). Reference numerals “129a” and “129b” denote connectors connecting the reaction chamber 100 with a supply tube 410a and an outlet tube 410b, respectively.

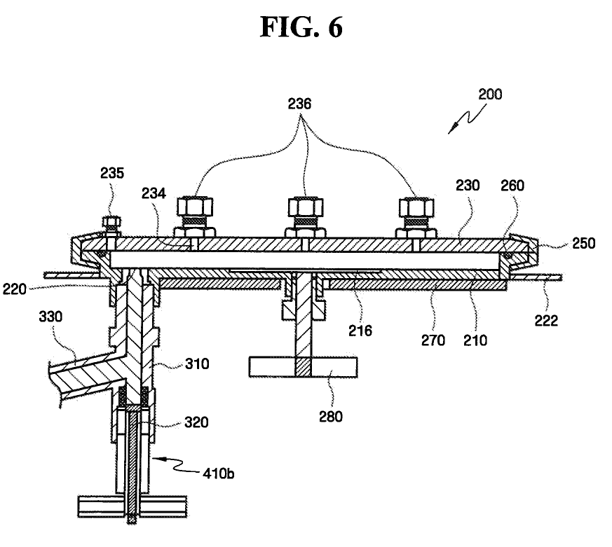

Hereinafter, a biopolymer synthesis apparatus according to a modified exemplary embodiment of the present invention will be described with reference to FIGS. 1 and 6. The biopolymer synthesis apparatus described with respect to FIGS. 1 and 6 is similar to the biopolymer synthesis apparatus described with respect to FIGS. 1 and 2, except with respect to the reaction chamber and outlet tube. Accordingly, the following explanation emphasizes different components of the reaction chamber and outlet tube shown in FIG. 6. An explanation of other components of the biopolymer synthesis apparatus will be omitted or briefly made.

FIG. 6 is a cross-sectional view of a modified exemplary reaction chamber according to an embodiment of the present invention.

Referring to FIGS. 1 and 6, a reaction chamber 200 may include a chamber body 210 and a chamber cover 230. The chamber cover 230 may be provided as a substantially flat plate or may be convexly shaped to increase an inner space of the reaction chamber 200. An O-ring 260 may be disposed between the chamber cover 230 and the chamber body 210 to increase a degree of sealing therebetween.

The chamber body 210 is coupled to the chamber cover 230 by a coupling means, such as a clamp 250. As described above, since the inside of the reaction chamber 200 is securely sealed by means of the chamber body 210, the chamber cover 230, the O-ring 260 and the clamp 250, it is possible to prevent contaminants from infiltrating through a crevice between the chamber body 210 and the chamber cover 230 during biopolymer synthesis.

The chamber cover 230 includes at least one through hole 234 formed through its top surface. The through hole 234 spatially connects the inner space of the reaction chamber 200 with the outside of the reaction chamber 200. First and second connectors 235 and 236, respectively, are fastened to the top surface of the chamber cover 230 having the at least one through hole 234. However, there is no particular limitation to the number and arrangement of through holes 234 and the first and second connectors 235 and 236.

A stage 216, on which a substrate where biopoloymers are to be synthesized can be seated, is provided at a central portion of the chamber body 210. The stage 216 is connected to a lower portion of a handle 280 for moving the stage 216. For example, the stage 216 may be installed to be movable up and down such that, if the handle 280 rotates clockwise, the stage 216 moves upward and if the handle 280 rotates counterclockwise, the stage 216 moves downward. A support plate 222 may be provided in the vicinity of the chamber body 210. The support plate 222 may be detachably coupled to support edges of the chamber body 210. The support plate 222 may be coupled to a lower support stand (not shown) by means of a fixing screw (not shown) and the chamber body 210 can be securely fixed to the support stand accordingly.

A heater 270 for heating the inside of the reaction chamber 200 may be provided at a bottom surface of the chamber body 210. For example, the heater 270 may include an electric heater to control the reaction temperature of the reaction chamber 200. It may be advantageous to provide the heater 270 as an electric heater because it can be easily switched on/off and the temperature can be easily controlled.

As illustrated in FIG. 6, an outlet tube 410b includes a first sub-outlet tube 310 and a second sub-outlet tube 330 branched from the first sub-outlet tube 310. The first sub-outlet tube 310 is connected to an outlet hole 220 of the chamber body 210 and incorporates a piston 320. The piston 320 may be moved up and down within the first sub-outlet tube 310 to control the spatial connection between the outlet hole 220 and/or the first sub-outlet tube 310 and the second sub-outlet tube 330. For example, if a head of the piston 320 moves upward to an upper region of the branched portion of the second sub-outlet tube 330, then the second sub-outlet tube 330 is closed. Accordingly, a reagent or a cleaning solution is not discharged from the chamber 200. However, if the head of the piston 320 moves down to a lower region of the branched portion of the second outlet tube 330, then the outlet hole 220 and/or the first outlet tube 310 and the second outlet tube 330 are spatially connected with each other so that the reagent or the cleaning solution can be discharged from the reaction chamber 200.

According to the biopolymer synthesis apparatus described above with respect to FIGS. 1 and 6, different biopolymer synthesis reagents can be easily recovered.

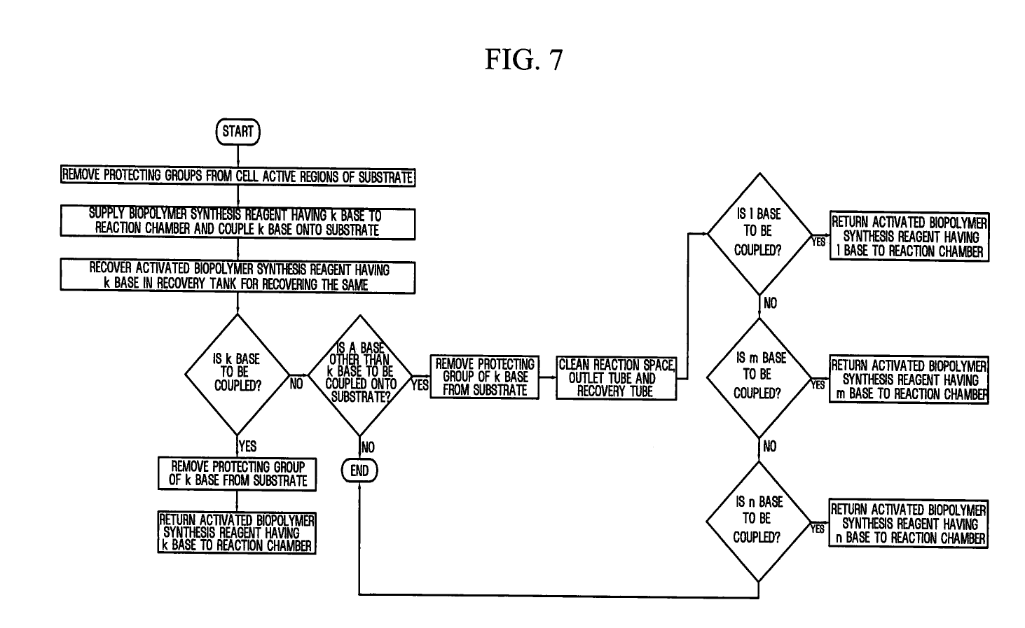

A method of synthesizing a biopolymer according to an embodiment of the present invention will be described in detail with reference to FIGS. 7 through 14.

FIG. 7 is a flowchart illustrating a method of synthesizing a biopolymer according to an embodiment of the present invention.

Referring to FIG. 7, protecting groups are first removed from the substrate having cell active regions. Then, a first biopolymer synthesis reagent, a cleaning solution and an activator are supplied to a reaction chamber in which the substrate is seated, and the biopolymer synthesis reagent is then synthesized on the substrate.

As described above, the biopolymer synthesis reagent may be a nucleotide phophoramidite monomer having a base that is one of Adenine (A), Thymine (T), Guanine (G), Cytosine (C) and Uracil (U). In one embodiment, the biopolymer synthesis reagent may be a deoxyribonucleoside phophoramidite monomer having photolabile or acid labile protecting groups coupled thereto. For example, the first biopolymer synthesis reagent may be a deoxyribonucleoside phophoramidite monomer having a k base, e.g., Adenine (A). The deoxyribonucleoside phophoramidite monomer is dissolved in a cleansing solution such as acronitrile and the resulting mixture can be reacted with a substrate. The deoxyribonucleoside phophoramidite monomer may have a concentration ranging from about 0.1 mM to about 500 mM.

Next, if there are any reagents other than the first biopolymer synthesis reagent remaining in the outlet tubes, the stuck reagents may be cleaned using a cleaning solution. Subsequently, the first biopolymer synthesis reagent remaining in the reaction chamber is recovered in a first recovery tank selected from a plurality of recovery tanks via the outlet tube.

Next, the reaction space and the outlet tube with the remaining first biopolymer synthesis reagent can be cleaned using the cleaning solution.

Then, a second biopolymer synthesis reagent is supplied to the reaction chamber in which the substrate is seated, and the second biopolymer synthesis reagent is then synthesized on the substrate.

The second biopolymer synthesis reagent remaining in the reaction chamber is recovered in a second recovery tank selected from the plurality of recovery tanks via the outlet tube. The second biopolymer synthesis reagent may be a deoxyribonucleoside phophoramidite monomer having a base different from that of the first biopolymer synthesis reagent, e.g., Cytosine (C). Similarly, four biopolymer synthesis reagents having different bases can be recovered in the respective recovery tanks.

In other words, when first biopolymer synthesis reagent having k base has been cleaned, it is determined whether the k base is to be coupled onto the substrate. If it is determined that the k base is to be coupled onto the substrate, the protecting group of the k base is removed from the substrate. A recycled biopolymer synthesis reagent having the activated k base is returned to reaction chamber from the first (k^{th}) recovery tank. If it is not necessary to couple k bases onto the surface of the substrate, it is determined which base among the bases l, m and n is to be coupled. Specifically, the protecting group of the k base is removed from the substrate. Then, the reaction space, outlet tubes, recovery tubes may be cleaned. If it is determined that the l base (or, m base or n base) is to be coupled onto the substrate, a recycled biopolymer synthesis reagent having the activated l base (or, m base or n base) is returned to reaction chamber from the second (l^{th}, or m^{th} or n^{th}) recovery tank.

The method of synthesizing a biopolymer may further comprise returning the biopolymer synthesis reagent to the substrate for coupling bases on a surface of the substrate, after the synthesizing and recovering cycle using the first biopolymer synthesis reagent, and coupling the k base onto the substrate.

Thereafter, it is determined whether the k base or a base other than the k base is to be coupled onto the substrate.

If it is determined that the k base is to be coupled onto the substrate, the protecting group of the k base is removed from the substrate and the activated first biopolymer synthesis reagent having the k base is returned from a recovery tank (recovery tank for k base) for recovering the biopolymer synthesis reagent having the k base to the reaction chamber and coupling of the k base onto the substrate occurs again. Upon returning the first biopolymer synthesis reagent, the first biopolymer synthesis reagent is pumped using a return pump.

If it is determined that the k base is not to be coupled onto the substrate, it is determined whether a base other than the k base is to be coupled onto the substrate. According to a determination result, if there are no further bases to be coupled, the biopolymer synthesis cycle is terminated. If there are additional bases to be coupled, the protecting group having a k base is removed from the substrate. Next, the reaction space of the reaction chamber, the outlet tube, the recovery tubes and the return tubes are cleaned using a cleaning solution.

Subsequently, it is determined which base among the bases l, m and n is to be coupled, and a biopolymer synthesis reagent having the base l, m or n to be coupled is subjected to the same synthesis, recovery and returning cycles as the biopolymer synthesis reagent having the k base was.

The plurality of recovery tanks may comprise a molecular sieve provided to remove moisture contained in the activated biopolymer synthesis reagent. The molecular sieve increases the storage stability of the activated biopolymer synthesis reagent, and thereby increases the ability of the biopolymer synthesis reagent to be recycled. As described above, the biopolymer synthesis reagents having different bases are recovered in different recovery tanks, respectively, and a cleaning cycle is performed at the start of each new cycle for recycling the biopolymer synthesis reagents, which is economically efficient. Considering that only about 10% of the biopolymer synthesis reagent injected into the reaction chamber is utilized in the reaction, the economic efficiency is high. In particular, since the amidite-based reagent is commercially available at high costs, the economic efficiency is much higher.

The synthesis and recovery cycles of the first and second biopolymer reagents are performed two or more times, respectively. The first biopolymer synthesis reagent produced after the first cycle of the two or more synthesis and recovery cycles is the first biopolymer synthesis reagent returned from the first recovery tank. The second biopolymer synthesis reagent produced after the first cycle of the two or more synthesis and recovery cycles is the second biopolymer synthesis reagent returned from the second recovery tank. Accordingly, after a biopolymer synthesis reagent having a particular base is supplied to the reaction and subsequently returned, the recycled biopolymer synthesis reagent in the recovery tank can be utilized for coupling bases until the purity and concentration of the activated biopolymer synthesis reagent is low.

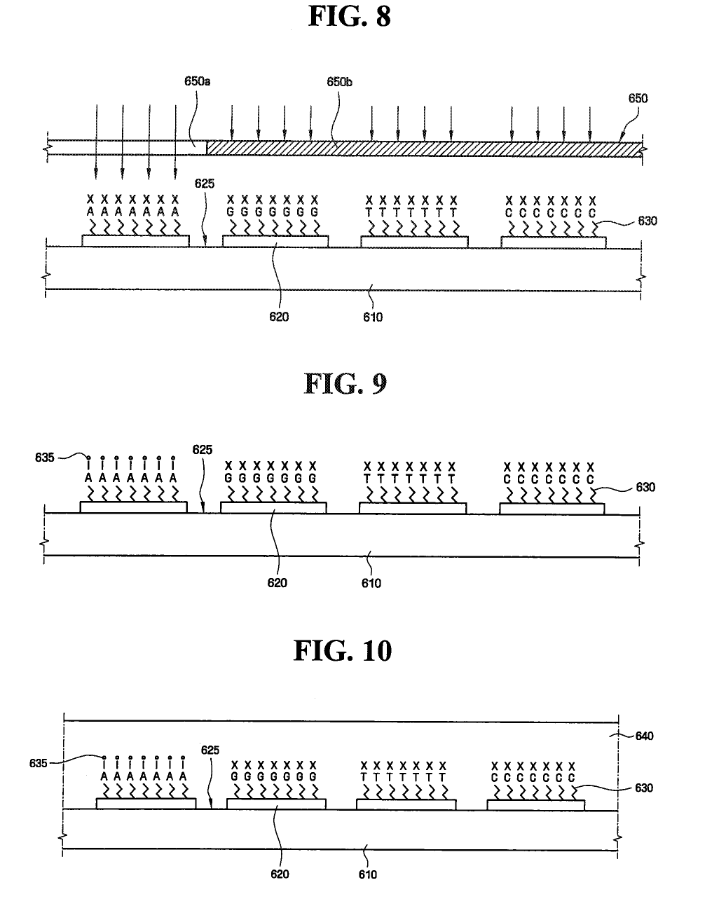

FIGS. 8 through 11 are cross-sectional views illustrating the processing steps of a biopolymer synthesis method according to an embodiment of the present invention.

FIGS. 8 and 9 illustrate wherein monomers have been coupled to a substrate 610 for biopolymer synthesis, and a step of exposing a functional group that can be coupled to a biopolymer.

Referring to FIG. 8, according to a biopolymer synthesis method of the present embodiment, a substrate 610 onto which a plurality of monomers are to be coupled is prepared. In one embodiment, each monomer may be a nucleotide phophoramidite monomer having a base that is Adenine (A), Guanine (G), Thymine (T), Cytosine (C), or Uracil (U). Each monomer contains a functional group (e.g., 635 in FIG. 9) that can be coupled to another monomer that is protected by a photolabile protecting group X. Examples of the functional group 635 may include hydroxyl, aldehyde, carboxyl, amide, thiol, halogen and sulfonate groups.

A plurality of cell active regions 620 are formed on the substrate 610. A plurality of cell separation regions 625 physically and/or chemically separate the plurality of cell active regions from one another. The cell active regions 620 may, for example, include a silicon oxide layer such as a plasma enhanced tetra-ethyl ortho silicate (PE-TEOS) layer, a high-density plasma (HDP) oxide layer, a P-SiH₄ oxide layer, or a thermal oxide layer; a silicate such as hafnium (Hf) silicate or zirconium (Zr) silicate; a metal oxynitride layer such as a Si oxynitride layer, a Hf oxynitride layer, or a Zr oxynitride layer; a metal oxide layer such as a titanium (Ti) oxide layer, a tantalum (Ta) oxide layer, an aluminum (Al) oxide layer, a Hf oxide layer, a Zr oxide layer, or an indium tin oxide (ITO) layer; a metal such as gold, silver, copper, or palladium (Pa); or a polymer such as polyimide, polyamine, polystyrene, polyacrylic acid, or polyvinyl; or the like or a combination thereof. In one embodiment, monomers can be coupled directly to the cell active regions 620. In another embodiment, monomers can be indirectly coupled to the cell active regions 620 via linkers 630.

Subsequently, a mask 650 having a light transmissive area 650a and a light blocking area 650b is used to selectively expose a cell active region 620.

Referring to FIG. 9, photolabile protecting groups X coupled to corresponding monomers are removed from a cell active region 620 exposed by the mask 650. As a result, the functional group 635 that can be coupled to another monomer is exposed.

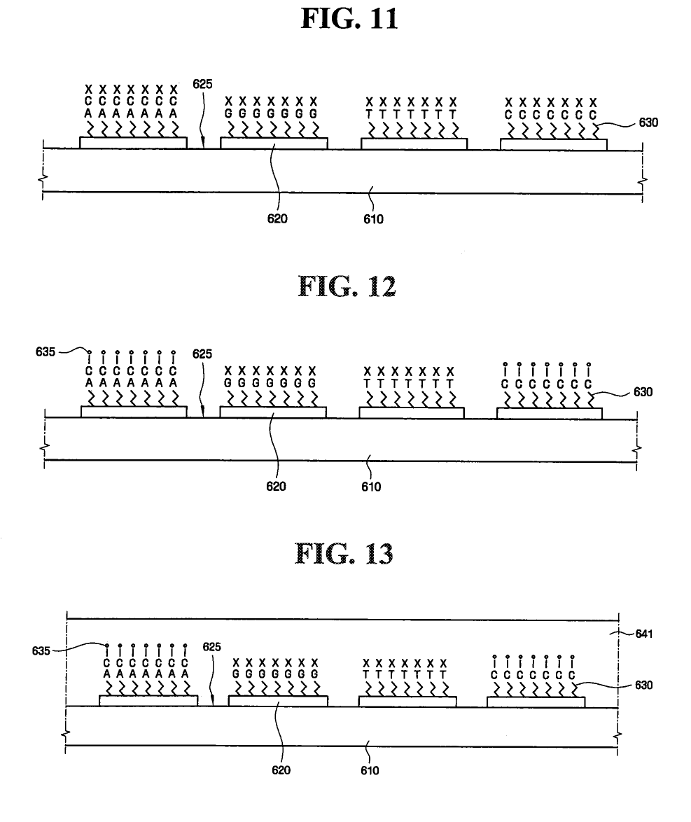

FIGS. 10 and 11 illustrate the step of synthesizing a biopolymer on a substrate.

Referring to FIG. 10, a biopolymer synthesis reagent 640 is provided onto the resulting structure shown in FIG. 9. In the illustrated embodiment, the biopolymer synthesis reagent 640 is a nucleotide phophoramidite monomer CX, having a base of Cytosine (C) protected by the photolabile protecting group X. The biopolymer synthesis reagent 640 selectively reacts with only the monomer having the exposed functional group 635 that can be coupled to another monomer (e.g., monomer A, as illustrated).

Referring to FIG. 11, the biopolymer synthesis reagent 640 is removed and two monomers A and CX are coupled together on a specific cell active region 620 to form a biopolymer ACX.

Referring to FIG. 12, to allow a recycled biopolymer synthesis reagent (e.g., 641 of FIG. 13) to react with a substrate 610, functional groups 635 having the protecting groups X are exposed to remove the protecting groups X and enable coupling to other monomers. In the illustrated embodiment, functional groups 635 in two or more cell active regions 620 can be simultaneously exposed.

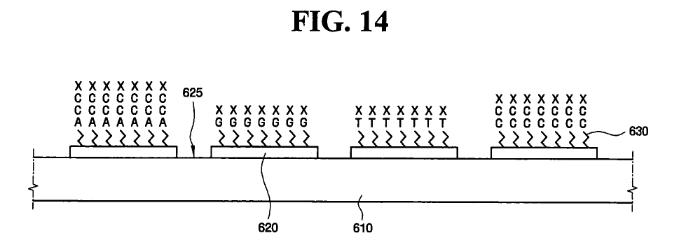

FIGS. 13 and 14 illustrate the step of synthesizing a biopolymer on a substrate using a recycled biopolymer synthesis reagent.

Referring to FIG. 13, a recycled biopolymer synthesis reagent 641 is provided onto the resulting structure shown in FIG. 12. In the illustrated embodiment, the recycled biopolymer synthesis reagent 641 can be a nucleotide phophoramidite monomer CX, having a base of Cytosine (C) protected by the photolabile protecting group X. The recycled biopolymer synthesis reagent 641 selectively reacts with only monomers having exposed functional groups 635 that can be coupled to other monomers.

Referring to FIG. 14, the recycled biopolymer synthesis reagent 641 is removed, thereby forming a coupled biopolymer ACCX having AC and CX coupled to each other and a coupled biopolymer CCX having C and CX coupled to each other are formed on specific cell active regions 620. In the illustrated embodiment, the recycled biopolymer synthesis reagent 641 passes through an outlet tube, a return tube, etc., which are cleaned by a cleaning solution, and then stored in a recovery tank before being returned to the reaction chamber, thereby maintaining high purity and high concentration of the recycled biopolymer synthesis reagent 641.

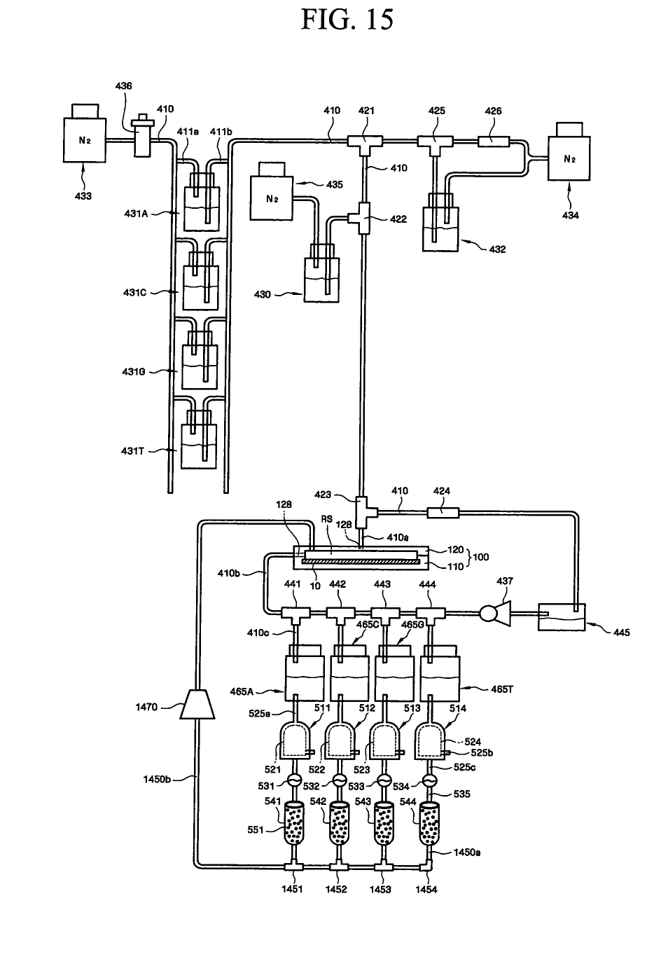

Hereinafter, a biopolymer synthesis apparatus according to another embodiment of the present invention will be described with reference to FIG. 15. In the embodiment shown in FIG. 15, substantially the same components as those described with respect to FIGS. 1 and 2 are identified by the same reference numerals and their repetitive description will be omitted or briefly made.

Referring to FIG. 15, an apparatus of synthesizing a biopolymer according to another embodiment of the present invention may, for example, include a plurality of recovery tanks (e.g., first recovery tank 465A, second recovery tank 465C, third recovery tank 465G and fourth recovery tank 465T) connected to a plurality of filtering devices (e.g., first filtering device 511, second filtering device 512, third filtering device 513 and fourth filtering device 514), rather than being directly connected to a return tube 450b as shown in FIG. 1. Each of the recovery tanks 465A, 465C, 465G and 465T contains a recycling agent used for recycling a biopolymer synthesis reagent. The activated biopolymer synthesis reagent in the reaction chamber 100 is converted into a recycled biopolymer synthesis reagent. For example, if a deoxyribonucleoside phophoramidite monomer and a 1H-tetrazole activator are supplied to the reaction chamber 100 for biopolymer synthesis, the deoxyribonucleoside phophoramidite activated monomer in the reaction chamber 100 is recovered as a deoxyribonucleoside phophoramidite monomer using excessive N, N-diisopropylamine. Mixtures containing deoxyribonucleoside phophoramidite monomers and N, N-diisopropylammonium tetrazolid salts may coexist in the recovery tanks 465A, 465C, 465G and 465T, respectively.

The plurality of filtering devices 511, 512, 513 and fourth 514 are connected to corresponding ones of the recovery tanks 465A, 465C, 465G and 465T, respectively, via recovery tubes 525a and filter the deoxyribonucleoside phophoramidite monomer from the mixtures by means of filters (e.g., first filter 521, second filter 522, third filter 523 and fourth filter 524). Meanwhile, N, N-diisopropylammonium tetrazolid salts are discharged to intermediate return tubes 525b to be recycled.

The filtered deoxyribonucleoside phophoramidite monomers are purified by a plurality of purifiers (e.g., first purifier 541, second purifier 542, third purifier 543 and fourth purifier 544) connected to corresponding ones of the filtering devices 511, 512, 513 and 514, thereby achieving a desired level of purity. In the purifying cycle using the respective purifiers 541, 542, 534 and 544, chromatography using silica gel 551 can be used. The purity and concentration of the purified recycled biopolymer synthesis reagent are compared with those of the biopolymer synthesis reagent. According to a comparison result, if the purity and concentration of the recycled biopolymer synthesis reagent resulting after the purifying cycle are not lower than those of the biopolymer synthesis reagent, the recycled biopolymer synthesis reagent can be introduced into the reaction chamber 100 to be recycled. The concentration of the recycled biopolymer synthesis reagent can be identified through comparison with the biopolymer synthesis reagent using HPLC (High Performance Liquid Chromatography). The purity of the recycled biopolymer synthesis reagent can be measured by P-NMR.

Although not shown, intermediate return tubes 525b connected to each of the filtering devices 511, 512, 513 and 514 may be connected to sub-return tubes 1450a connected to corresponding ones of the purifiers 541, 542, 543 and 544.

Meanwhile, the recycled biopolymer synthesis reagent can be evaporated by evaporators (e.g., first evaporator 531, second evaporator 532, third evaporator 533 and fourth evaporator 534) disposed between corresponding ones of the filtering devices 511, 512, 513 and 514 and purifiers 541, 542, 543 and 544, thereby stabilizing the recycled biopolymer synthesis reagent. The evaporators 531, 532, 533 and 534 may further include a molecular sieve. In one embodiment, the evaporators 531, 532, 533 and 534 may be connected to corresponding ones of the filtering devices 511, 512, 513 and 514 via evaporator input tubes 525c. In one embodiment, the evaporators 531, 532, 533 and 534 may be connected to corresponding ones of the purifiers 541, 542, 543 and 544 via evaporator output tubes 535.

The recycled biopolymer synthesis reagent is led to a return tube 1450b via sub-return tubes 1450a connected to the respective purifiers 541, 542, 543 and 544. A return pump 1470 may be provided at the return tube 1450b for pumping the recycled biopolymer synthesis reagent to the reaction chamber 100. In the illustrated embodiment, different recycled biopolymer synthesis reagents may be returned by adjusting passageways using a plurality of return valves (e.g., first return valve 1451, second return valve 1452, third return valve 1453 and fourth return valve 1454).

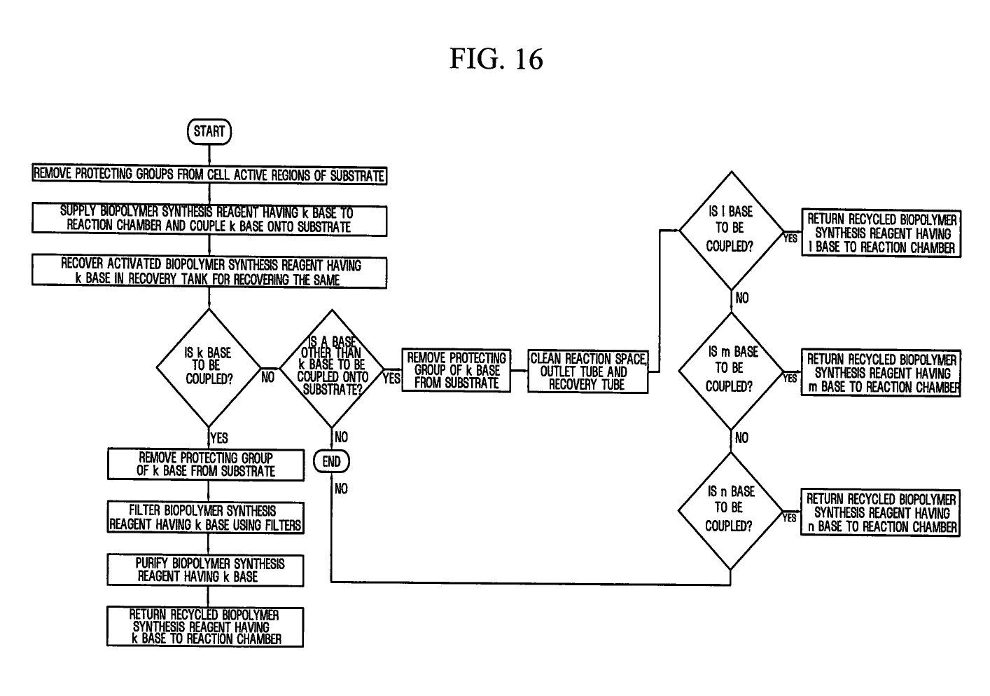

A method of synthesizing a biopolymer according to another embodiment of the present invention will be described in detail with reference to FIG. 16.

FIG. 16 is a flowchart illustrating a method of synthesizing a biopolymer according to another embodiment of the present invention.

Similar to the method of synthesizing a biopolymer described with respect to FIG. 7, protecting groups are first removed from the substrate having cell active regions, a biopolymer synthesis reagent having a k base is provided and k bases are coupled onto the substrate using the biopolymer synthesis reagent. The first biopolymer synthesis reagent having k base is recovered in a first (k^{th}) recovery tank selected from a plurality of recovery tanks via the outlet tube.

Next, it is determined whether the k base is to be coupled onto the substrate. If it is determined that the k base is to be coupled onto the substrate, the protecting group of the k base is removed from the substrate. A recycled biopolymer synthesis reagent having the k base, which has been recovered from the reaction chamber, is filtered. Accordingly, the recycled biopolymer synthesis reagent having the k base (e.g., a deoxyribonucleoside phophoramidite monomer) is isolated from a mixture containing deoxyribonucleoside phophoramidite monomers and N, N-diisopropylammonium tetrazolid salts using filters.

Next, the recycled biopolymer synthesis reagent having the k base is purified. Thereafter, the purity and concentration of the recycled biopolymer synthesis reagent are measured. Then, if it is necessary to couple k bases onto a surface of the substrate, the recycled biopolymer synthesis reagent having the k base is returned to the reaction chamber.

If it is not necessary to couple k bases onto the surface of the substrate, the protecting group of the k base is removed from the substrate. Then, the reaction space, outlet tubes, recovery tubes may be cleaned, and it is determined which base among the bases l, m and n is to be coupled. Then, the recycled biopolymer synthesis reagent having the base to be coupled is subjected to the same synthesis, recovery and returning cycles as the of biopolymer synthesis reagent having the k base was.

The synthesizing, filtering and purifying cycles of the biopolymer synthesis reagent can reduce waste in the reagents.

In biopolymer synthesis apparatuses, methods thereof, and methods of recovering reagents for synthesizing biopolymers according to some embodiments of the present invention, different biopolymer synthesis reagents with high purity and high concentration can be recovered to be recycled. Since the biopolymer synthesis reagent can be recycled, biopolymers can ultimately be synthesized at lower costs. In addition, since high-purity biopolymer synthesis reagents can be recovered, environmental contamination due to biopolymer synthesis reagent wastes can be prevented.

Embodiments of the present invention may be practiced in many ways. What follows in the paragraphs below is a discussion of some exemplary embodiments of the present invention.

One exemplary embodiment can be generally characterized as a method of recovering a biopolymer synthesis reagent that includes: supplying a first biopolymer synthesis reagent to a reaction chamber in which a substrate is seated and synthesizing the first biopolymer synthesis reagent on the substrate, wherein an amount of first biopolymer synthesis reagent remains within the reaction chamber; recovering the amount of first biopolymer synthesis reagent in a first recovery tank via an outlet tube; cleaning the reaction chamber and the outlet tube using cleaning solution; supplying a second biopolymer synthesis reagent to the reaction chamber and synthesizing the second biopolymer synthesis reagent on the substrate, wherein an amount of second biopolymer synthesis reagent remains within the reaction chamber; and recovering the amount of second biopolymer synthesis reagent in a second recovery tank of the plurality of recovery tanks via the outlet tube.

In one embodiment, the first and second biopolymer synthesis reagents may include deoxyribonucleoside phophoramidite reagents having different bases.

In one embodiment, the first and second reagents may have a base that includes one of Adenine (A), Thymine (T), Guanine (G), Cytosine (C) and Uracil (U).

In one embodiment, the aforementioned method may further include cleaning the outlet tube using a cleaning solution after synthesizing the first biopolymer synthesis reagent and before recovering the first biopolymer synthesis reagent.

Another exemplary embodiment can be generally characterized as a method of recovering a biopolymer synthesis reagent that includes: performing a first biopolymer synthesis and recovery cycle; performing a second biopolymer synthesis and recovery cycle; and performing a cleaning cycle after the first biopolymer synthesis and recovery cycle and before the second biopolymer synthesis and recovery cycle. The first biopolymer synthesis and recovery cycle may include: supplying a first biopolymer synthesis reagent to a reaction chamber in which a substrate is seated and synthesizing the first biopolymer synthesis reagent on the substrate, wherein an amount of first biopolymer synthesis reagent remains within the reaction chamber; and recovering the amount of first biopolymer synthesis reagent in a first recovery tank via an outlet tube. The second biopolymer synthesis and recovery cycle may include: supplying a second biopolymer synthesis reagent to a reaction chamber in which a substrate is seated and synthesizing the second biopolymer synthesis reagent on the substrate, wherein an amount of second biopolymer synthesis reagent remains within the reaction chamber; and recovering the amount of second biopolymer synthesis reagent in a second recovery tank via the outlet tube. The cleaning cycle may include cleaning the reaction chamber and the outlet tube using a cleaning solution. Each of the first and second biopolymer synthesis and recovery cycles may be performed two or more times. The first biopolymer synthesis reagent produced after the first cycle of the two or more first synthesis and recovery cycles may include the first biopolymer synthesis reagent returned from the first recovery tank and wherein the second biopolymer synthesis reagent produced after the first cycle of the two or more second synthesis and recovery cycles includes the second biopolymer synthesis reagent returned from the second recovery tank.

In one embodiment, the cleaning cycle may be performed by a process that includes cleaning a return tube returning the first and second biopolymer synthesis reagents.

In one embodiment, the aforementioned method may further include returning the first and second biopolymer synthesis reagents by pumping the first and second biopolymer synthesis reagents using a return pump.

In one embodiment, the aforementioned method may further include filtering the first and second biopolymer synthesis reagents before returning the first and second biopolymer synthesis reagents.

In one embodiment, the aforementioned method may further include purifying the first and second biopolymer synthesis reagents after filtering the first and second biopolymer synthesis reagents.

In one embodiment, the aforementioned method may further include evaporating the first and second biopolymer synthesis reagents after filtering the first and second biopolymer synthesis reagents and before purifying the first and second biopolymer synthesis reagents using a plurality of evaporators.

In one embodiment, the first and second biopolymer synthesis reagents include first and second amidite-based reagents.

In one embodiment, the first and second amidite-based reagents include deoxyribo nucleoside phophoramidite reagents having different bases.

While embodiments of the present invention have been exemplarily shown and described above, it will be understood by those of ordinary skill in the art that various changes in form and details may be made therein without departing from the spirit and scope of the present invention as defined by the following claims. It is therefore desired that the present embodiments be considered in all respects as illustrative and not restrictive, reference being made to the appended claims rather than the foregoing description to indicate the scope of the invention.

What is claimed is:

1. An apparatus for synthesizing a biopolymer comprising:
a reaction chamber;
an outlet tube connected to the reaction chamber;
a plurality of recovery tanks connected to the outlet tube; and
a plurality of recovery valves configured to open or block passageways between the outlet tube and corresponding ones of the recovery tanks.

2. The apparatus of claim 1, further comprising a plurality of recovery tubes branched from the outlet tube and connected to corresponding ones of the plurality of recovery tanks, wherein the plurality of recovery valves are connected to corresponding ones of the plurality of recovery tubes.

3. The apparatus of claim 1, further comprising:
a plurality of reagent tanks connected to the reaction chamber and structured to provide different reagents; and
a cleaning tank connected to the reaction chamber and structured to provide a cleaning solution to the outlet tube.

4. The apparatus of claim 3, wherein the reaction chamber is structured to receive the different reagents from the plurality of reagent tanks, the outlet tube is structured to discharge the different reagents from the reaction chamber, and the plurality of recovery valves are structured to introduce the different reagents discharged from the reaction chamber to corresponding ones of the plurality of reagent tanks.

5. The apparatus of claim 1, further comprising:
a plurality of sub-return tubes connected to corresponding ones of the plurality of reagent tanks; and
a return tube connecting the plurality of sub-return tubes with the reaction chamber, wherein the plurality of sub-return tubes are branched from the return tube.

6. The apparatus of claim 5, further comprising:
a plurality of return valves connected to corresponding ones of the plurality of sub-return tubes; and
a return pump connected to the return tube.

7. The apparatus of claim 1, further comprising a shaking apparatus for shaking the reaction chamber.

8. The apparatus of claim 1, wherein the reaction chamber comprises:
a chamber body; and
a chamber cover coupled to the chamber body to provide a sealed reaction space,
wherein the outlet tube is connected to the reaction chamber.

9. The apparatus of claim 8, wherein the outlet tube comprises:
a first sub-outlet tube directly connected to the chamber body;
a second sub-outlet tube branched from the first sub-outlet tube and connected to the plurality of recovery tanks; and
a piston in the first sub-outlet tube and configured to control a spatial connection between the first sub-outlet tube and second sub-outlet tube.

10. The apparatus of claim 1, further comprising a plurality of filtering devices connected to corresponding ones of the plurality of recovery tanks and configured to filter reagents discharged from the corresponding ones of the plurality of recovery tanks.

11. The apparatus of claim 10, further comprising a plurality of purifiers connected to corresponding ones of the plurality of filtering devices and configured to purify reagents discharged from corresponding ones of the plurality of filtering devices.

12. The apparatus of claim 11, further comprising a plurality of evaporators disposed between corresponding ones of the filtering devices and the plurality of purifiers, the plurality of evaporators configured to evaporate reagents discharged from corresponding ones of the plurality of filtering devices.

13. A method of recovering a biopolymer synthesis reagent comprising:
supplying a first biopolymer synthesis reagent to a reaction chamber in which a substrate is seated and synthesizing the first biopolymer synthesis reagent on the substrate, wherein an amount of first biopolymer synthesis reagent remains within the reaction chamber;
recovering the amount of first biopolymer synthesis reagent in a first recovery tank via an outlet tube;
cleaning the reaction chamber and the outlet tube using cleaning solution;
supplying a second biopolymer synthesis reagent to the reaction chamber and synthesizing the second biopolymer synthesis reagent on the substrate, wherein an amount of second biopolymer synthesis reagent remains within the reaction chamber; and
recovering the amount of second biopolymer synthesis reagent in a second recovery tank via the outlet tube.

14. The method of claim 13, wherein the first and second biopolymer synthesis reagents include deoxyribonucleoside phophoramidite reagents having different bases.

15. The method of claim 14, wherein the first and second reagents have a base that includes one of Adenine (A), Thymine (T), Guanine (G), Cytosine (C) and Uracil (U).

16. The method of claim 13, further comprising cleaning the outlet tube using a cleaning solution after synthesizing the first biopolymer synthesis reagent and before recovering the first biopolymer synthesis reagent.

17. A method of recovering a biopolymer synthesis reagent comprising:
performing a first biopolymer synthesis and recovery cycle comprising:
supplying a first biopolymer synthesis reagent to a reaction chamber in which a substrate is seated and synthesizing the first biopolymer synthesis reagent on the substrate, wherein an amount of first biopolymer synthesis reagent remains within the reaction chamber; and
recovering the amount of first biopolymer synthesis reagent in a first recovery tank selected from a plurality of recovery tanks via an outlet tube;
performing a second biopolymer synthesis and recovery cycle comprising:
supplying a second biopolymer synthesis reagent to a reaction chamber in which a substrate is seated and synthesizing the second biopolymer synthesis reagent on the substrate, wherein an amount of second biopolymer synthesis reagent remains within the reaction chamber; and
recovering the amount of second biopolymer synthesis reagent in a second recovery tank selected via the outlet tube; and
performing a cleaning cycle after the first biopolymer synthesis and recovery cycle and before the second biopolymer synthesis and recovery cycle, wherein the cleaning cycle comprises cleaning the reaction chamber and the outlet tube using a cleaning solution,
wherein each of the first and second biopolymer synthesis and recovery cycles are performed two or more times, wherein the first biopolymer synthesis reagent produced after the first cycle of the two or more first synthesis and recovery cycles includes the first biopolymer synthesis reagent returned from the first recovery tank and wherein the second biopolymer synthesis reagent produced after the first cycle of the two or more second synthesis and recovery cycles includes the second biopolymer synthesis reagent returned from the second recovery tank.

18. The method of claim 17, wherein performing the cleaning cycle comprises cleaning a return tube returning the first and second biopolymer synthesis reagents.

19. The method of claim 17, further comprising returning the first and second biopolymer synthesis reagents by pumping the first and second biopolymer synthesis reagents using a return pump.

20. The method of claim 17, further comprising filtering the first and second biopolymer synthesis reagents before returning the first and second biopolymer synthesis reagents.

21. The method of claim 20, further comprising purifying the first and second biopolymer synthesis reagents after filtering the first and second biopolymer synthesis reagents.

22. The method of claim 21, further comprising evaporating the first and second biopolymer synthesis reagents after filtering the first and second biopolymer synthesis reagents and before purifying the first and second biopolymer synthesis reagents using a plurality of evaporators.

23. The method of claim 17, wherein the first and second biopolymer synthesis reagents include first and second amidite-based reagents.

24. The method of claim 23, wherein the first and second amidite-based reagents include deoxyribo nucleoside phophoramidite reagents having different bases.
